(19) United States
(12) Patent Application Publication
(10) Pub. No.: US 2026/0188449 A1
(43) Pub. Date: Jul. 2, 2026

(54) FINGERPRINTING OF UNSTRUCTURED PATIENT DATA

(71) Applicant: KONINKLIJKE PHILIPS N.V., EINDHOVEN (NL)

(72) Inventor: Eltjo Hans HASELHOFF, EINDHOVEN (NL)

(21) Appl. No.: 19/129,326

(22) PCT Filed: Nov. 29, 2023
(86) PCT No.: PCT/EP2023/083479

§ 371 Date: May 13, 2025

Related U.S. Application Data
(60) Provisional application No. 63/428,807, filed on Nov. 30, 2022.

(51) Int. Cl. G16H 15/00; G06N 3/0464; G16H 30/40

(52) U.S. Cl. CPC G16H 15/00; G06N 3/0464; G16H 30/40

(57) ABSTRACT

A convoluted neural network (CNN) for use in a medical workflow is generated by receiving a diagnostic indication, unstructured patient data (UPD), and a diagnostic report vocabulary list, wherein the diagnostic report vocabulary list comprises ordered vocabulary terms, generating a blank bitmap comprising a plurality of pixels having (i) a number of pixels corresponding to a quantity of the vocabulary terms and (ii) a correspondence between pixel positions in the bitmap and the order of the vocabulary terms, generating a fingerprint comprising a mapping of each occurrence of a vocabulary term in the UPD to a corresponding pixel position in the blank bitmap and training a convoluted neural network (CNN) using the fingerprint as an input and the diagnostic indication as a target output.

### BACKGROUND

Use of digital technologies in radiology has caused a substantial increase in the quantity of clinical image and non-image information used by healthcare professionals for diagnosis. Despite this growth, clinical assessments are still performed in the traditional fashion. This incongruence has caused large increases in workloads and data overload in healthcare professionals. Artificial intelligence (AI) methods with deep learning analysis through convoluted neural networks (CNNs) are being used with increased frequency to partially address the aforementioned problems in the field.

While AI offers one potential means of meeting the workload problem, the background information of a patient (e.g., non-image information, such as patient history, previous diagnostic reports etc.) remains a crucial component of reliable diagnosis. Finding appropriate patient information that is relevant to a specific clinical context remains a time-consuming task.

While AI may be used to find the appropriate patient information, unstructured patient data (UPD) must first be transformed into a structured format by using Natural Language Processing (NLP) or other methods. This is not a trivial task and automated NLP methods are still in their infancy. Errors generated by NLP during data structuring may introduce a cascade of new problems when the structured data is fed into an AI engine. In light of the current dynamics in healthcare worldwide (e.g., declining reimbursements, value-driven healthcare policies, shortage of medical staff worldwide and high rates of sick leave because of work stress), improvements to this data flow and structuring are urgently needed.

### SUMMARY

Some example embodiments are related to a method for receiving a diagnostic indication, unstructured patient data (UPD), and a diagnostic report vocabulary list, wherein the diagnostic report vocabulary list comprises ordered vocabulary terms, generating a blank bitmap comprising a plurality of pixels having (i) a number of pixels corresponding to a quantity of the vocabulary terms and (ii) a correspondence between pixel positions in the bitmap and the order of the vocabulary terms, generating a fingerprint comprising a mapping of each occurrence of a vocabulary term in the UPD to a corresponding pixel position in the blank bitmap and training a convoluted neural network (CNN) using the fingerprint as an input and the diagnostic indication as a target output.

Other example embodiments are related to a method for receiving unstructured patient data (UPD) and a diagnostic report vocabulary list, wherein the diagnostic report vocabulary list comprises ordered vocabulary terms, generating a blank bitmap comprising a plurality of pixels having (i) a number of pixels corresponding to a quantity of the vocabulary terms and (ii) a correspondence between pixel positions in the bitmap and the order of the vocabulary terms, generating a first fingerprint comprising a mapping of each occurrence of a vocabulary term in the UPD to a corresponding position in the blank bitmap by changing a pixel value associated with the corresponding pixel position in the bitmap and displaying the fingerprint to a user.

Other example embodiments are related to a system for creating a data structure for use in clinical diagnostic support, wherein such system comprises a memory including a diagnostic indication, unstructured patient data (UPD), and a diagnostic report vocabulary list, wherein the diagnostic report vocabulary list comprises ordered vocabulary terms. The system also comprises a processor configured to generate a blank bitmap comprising a plurality of pixels having (i) a number of pixels corresponding to a quantity of the vocabulary terms and (ii) a correspondence between pixel positions in the bitmap and the order of the vocabulary terms. The processor is also configured to generate a fingerprint comprising a mapping of each occurrence of a vocabulary term in the UPD to a corresponding pixel position in the blank bitmap and to train a convoluted neural network (CNN) using the fingerprint as an input and the diagnostic indication as a target output.

Other example embodiments are related to a system for creating a data structure for use in clinical diagnostic support, wherein such system comprises a memory including unstructured patient data (UPD) and a diagnostic report vocabulary list, wherein the diagnostic report vocabulary list comprises ordered vocabulary terms. The system also comprises a processor configured to generate a blank bitmap comprising a plurality of pixels having (i) a number of pixels corresponding to a quantity of the vocabulary terms and (ii) a correspondence between pixel positions in the bitmap and the order of the vocabulary terms. The processor is also configured to generate a first fingerprint comprising a mapping of each occurrence of a vocabulary term in the UPD to a corresponding position in the blank bitmap by changing a pixel value associated with the corresponding pixel position in the bitmap. The system also comprises a display for displaying the fingerprint to a user.

Other examples embodiments are related to a computer program products operable, when executed on a computer, to perform a method as described herein.

### BRIEF DESCRIPTION OF THE DRAWINGS

FIG. 1 shows an exemplary fingerprint reference matrix according to various exemplary embodiments.

FIG. 2 shows an unstructured fingerprint according to various exemplary embodiments.

FIG. 3 shows an unstructured fingerprint with frequently appearing entries labeled according to various exemplary embodiments.

FIG. 4 shows a differential fingerprint according to various exemplary embodiments.

FIG. 5 shows an unstructured fingerprint with an interactive word cloud according to various exemplary embodiments.

FIG. 6 shows a flow diagram for training a convoluted neural network using unstructured patient data and corresponding diagnostic reports according to various exemplary embodiments.

FIG. 7 shows a method diagram for training a convoluted neural network using unstructured patient data and corresponding diagnostic reports according to various exemplary embodiments.

FIG. 8 shows a vectorized unstructured fingerprint according to various exemplary embodiments.

FIG. 9 shows a flow diagram for use of an image-based convoluted neural network with a fingerprint-based convoluted neural network for use in clinical decision support according to various exemplary embodiments.

FIG. 10 shows a method diagram for use of an image-based convoluted neural network with a fingerprint-based convoluted neural network for use in clinical decision support according to various exemplary embodiments.

FIG. 11 shows a schematic drawing of an exemplary system according to various exemplary embodiments.

### DETAILED DESCRIPTION

The exemplary embodiments may be further understood with reference to the following description and the related appended drawings, wherein like elements are provided with the same reference numerals. The exemplary embodiments relate to use of a “fingerprint” to assist in both clinical and AI analysis of unstructured patient data (UPD).

As discussed above, use of Natural Language Processing (NLP) on UPD may introduce a litany of errors when AI analyzes the resulting structured patient data. The exemplary embodiments provide an alternative data structure for AI to analyze. This alternative data structure is termed a “fingerprint” throughout this description. A fingerprint may be utilized in clinical diagnostic support. At an elevated level of abstraction, a fingerprint may be understood to be an image-based representation of relevant clinical terminology (e.g., from a UPD source). It should be understood that the term fingerprint is used throughout this description to refer to a data structure that has certain characteristics as described herein. Thus, a fingerprint should be understood to be a data structure that has the characteristics as described herein.

Use of a fingerprint along with a corresponding clinical diagnostic report may be used to train a convoluted neural network (CNN), with the fingerprints as input data and the structured findings in the clinical report acting as the desired output. The fingerprint may take the form of a small (with respect to data use) greyscale image file, which may be analyzed by existing AI platforms for image-related training without modification. Once trained, a CNN may act as a “virtual NLP engine” and may be used to support an image-based CNN in a straightforward manner, using diagnostic images and the corresponding UPD.

The term virtual NLP is justified because an AI network may be trained to be triggered on the simultaneous occurrence of specific terms in the patient data, or specific words in a specific order, in case they relate to consistent clinical diagnostic findings. The AI may thus benefit from the analysis carried out by the healthcare professionals who created the diagnostic reports that were subsequently used for AI-training.

Another advantage of fingerprints is that they are small-sized images (ideally less than 100 kilobytes, though larger sizes are possible if an operator desires). This small size is independent of the amount of data the fingerprint represents, allowing for an efficient data format to store the essential aspects of UPD for use in NLP algorithms and longitudinal studies.

Fingerprints are useful for analysis of longitudinal studies. As will be described in greater detail below, fingerprints reveal any relevant differences between two large amounts of unstructured data without search and index algorithms, instead utilizing only image subtraction. The use of image subtraction reveals only the terminology which has changed between any two given studies for a given patient. Fingerprints may also be used as an attractive and efficient user interface (UI) visualization for user interaction with large amounts of UPD, to be described in greater detail below.

To create a fingerprint in a clinical context, various information and operations may be performed. This information and operations may include, for example, a dictionary (e.g., a medical dictionary), an extraction algorithm that creates a fingerprint from any source of unstructured, text-based data, and an image-driven AI engine that transforms the fingerprints to diagnostic suggestions for consideration by a clinician. A large collection of UPD along with related clinical diagnostic findings may be used to train the aforementioned AI engine.

Ideally, the information should be available in a longitudinal fashion (i.e., various versions of the same case/study over time). Use of longitudinal UPD and related clinical diagnostic findings may improve AI training efficiency. However, it should be understood that longitudinal data is not required.

As an example of using longitudinal data, two different data reports for a same patient may be considered. A first data report may include patient data created at time t₃ along with a latest diagnostic report for a clinical condition diagnosed shortly after time t₃. A second data report may include patient data created at time t₁ along with a diagnostic report created shortly after time t₁, plus the information created at time t₂ along with the diagnostic report created shortly after time t₂, plus the information created at time t₃ along with the diagnostic report created shortly after time t₃. The first data report will provide less information than the second data report because the incremental aspects of the information will be lost if all the data is bundled together.

The AI engine may be operated in parallel to an existing algorithm for an image-based AI, integrated with such an image-based AI engine (since the fingerprints are ordinary digital images), or as a stand-alone application for the generation of diagnostic suggestions obtained from the unstructured patient data during diagnosis.

As discussed above, to create a fingerprint, a dictionary may be used. It should be understood that the term “dictionary” encompasses any other representative word list as well. In an example, a medical dictionary containing 98,119 words is used as a fingerprint reference, along with 120 pages of PDF files to be used as the UPD.

Initially, a digital grayscale image of 314×314 one-byte pixels is created, leading to a total file size of 98.6 kilobytes. 314 is the smallest integer number the square of which is larger than the number of words in the dictionary being used as a fingerprinting reference ((3142=98,596)>98,119)).

Next, each pixel in the grayscale image is assigned one unique word from the dictionary. This operation may be performed in several ways, but in this example all pixels are filled from the top left to the bottom right of the grayscale image (i.e., reading order) with the words in the dictionary in alphabetical order. It should be understood that other mappings between the dictionary and the grayscale image are possible.

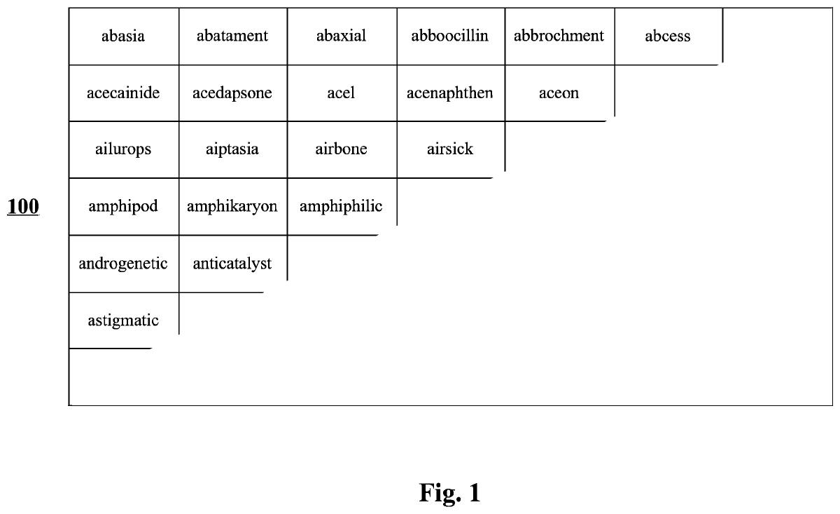

FIG. 1 shows an exemplary fingerprint reference matrix 100 according to various exemplary embodiments. FIG. 1 depicts the top left most corner of a 314×314 fingerprint reference matrix 100. The top left most corner is filled with the alphabetically first term of a corresponding dictionary (in this case, “abasia”). The matrix is filled alphabetically, left to right, row by row, until the dictionary is exhausted.

Use of a fingerprint reference matrix allows for each pixel in the 314×314 image to be uniquely assigned the n^{th} word in the dictionary by way of the following formula:

$$n = ( j - 1 ) , 314 + i , with 1 < i \cdot 314 and 1 < j \cdot 314$$

indicating that the n^{th} word of the dictionary will be assigned to the pixel at the i^{th} column from the right and the j^{th} row from the top.

With both the assignment formula and fingerprint reference matrix, creation of the fingerprint is straightforward. Beginning with the 314×314 image with all pixels set to value 0 (i.e., a uniformly black image), a standard text read algorithm reads each word from the unstructured data set (the aforementioned 120 pages of UPD PDFs). Each time a read word from the UPD is present in the fingerprint reference matrix, the corresponding pixel value of the fingerprint is increased by one. In this example, each pixel is bound to a maximum value of 255, corresponding to the maximum value of a single byte. While this maximum value is likely sufficient for all practical applications, it may be extended.

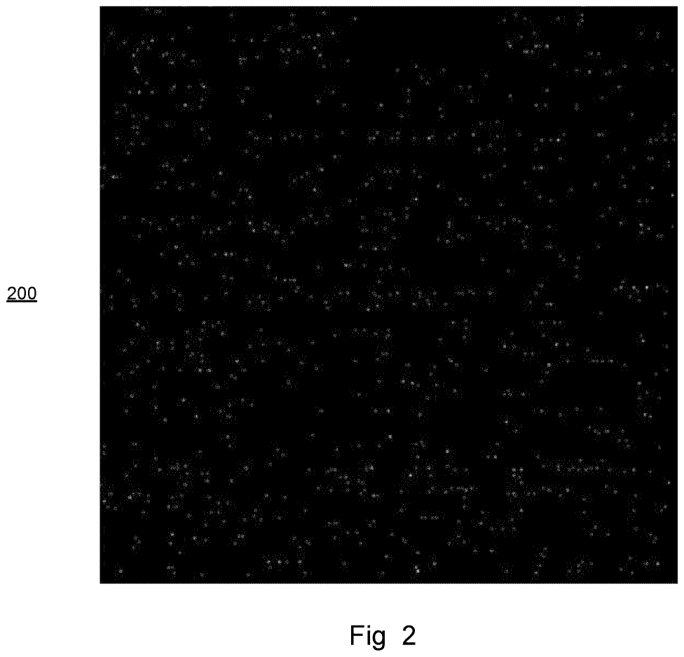

FIG. 2 shows an unstructured fingerprint 200 according to various exemplary embodiments. Of note in fingerprint 200 are the more prominent (brighter) pixels scattered throughout the image. The location of any given pixel corresponds to the n^{th} term in the dictionary being translated to the pixel grid top left to right, row by row. The brightness of any given pixel corresponds with the frequency that the n^{th} term in the dictionary appears in the UPD. Thus, more frequently appearing terms correspond to brighter pixels, up to and including a term that appears 255 times in the UPD (the maximum value of a byte).

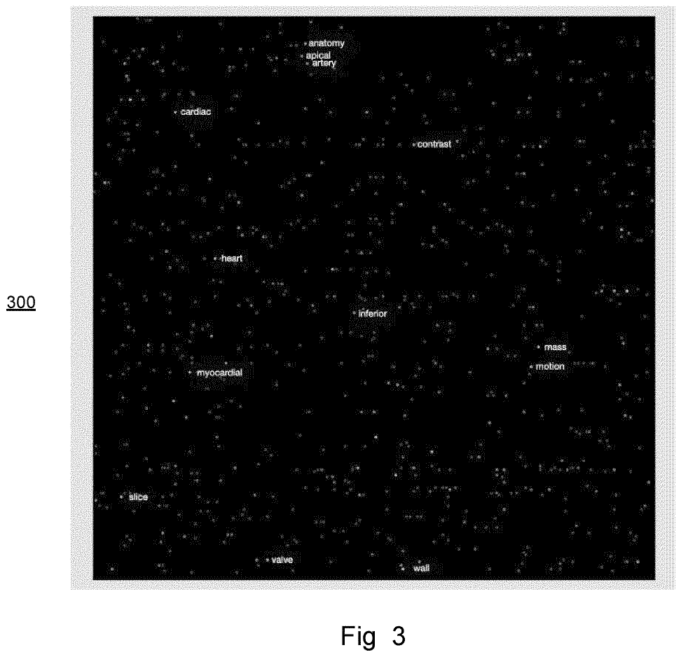

FIG. 3 shows an unstructured fingerprint 300 with frequently appearing entries labeled according to various exemplary embodiments. FIG. 3 depicts the same UPD as fingerprint 200 as shown in FIG. 2, but with frequently appearing terms (brighter pixels) labeled with their corresponding dictionary entry. The threshold for what constitutes a frequently appearing entry may be defined by an operator (e.g., 10 times, 50 times, 100 times, etc.). FIG. 3 shows the attractive means of visualizing UPD for a user.

It should be noted that a fingerprint can be an ambiguous identifier of the scanned UPD, because it is possible to create identical fingerprints with different data. However, in a clinical context, this ambiguity is irrelevant because clinicians are concerned with changes to the fingerprint (e.g., a longitudinal study). This clinical need is met because any change in the number of relevant terms (i.e., the dictionary terms used to populate the fingerprint reference matrix) will increase or decrease the brightness of the corresponding pixels in a fingerprint.

This property of fingerprints makes them attractive for use in longitudinal studies. Image subtraction of two fingerprints created at different dates will create a new, third, differential fingerprint, which reveals all relevant terms added at a second date. It should be understood that image subtraction may similarly reveal relevant terms deleted/removed from the second fingerprint as well.

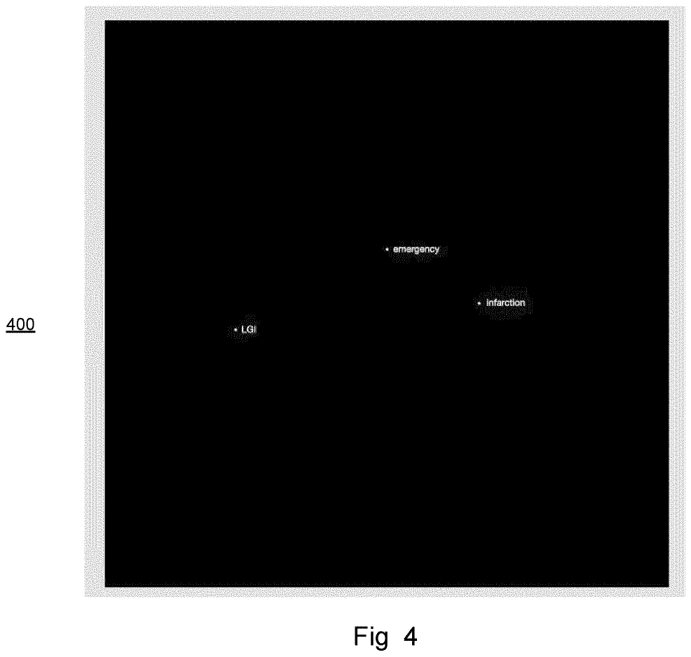

FIG. 4 shows a differential fingerprint 400 according to various exemplary embodiments. A differential fingerprint may be created by pixel-by-pixel image subtraction of two fingerprints created at different times (Fingerprint 2 “F2”-Fingerprint 1 “F1”). FIG. 4 does not depict the two fingerprints (F1, F2) used for its creation, because the relevant aspect is the depicted changes. Cursory analysis of differential fingerprint 400 reveals that F2 had data entered related to an emergency sudden cardiac infarction, diagnosed with a Late Gadolinium Enhancement (LGI) Magnetic Resonance Imaging (MRI) scan. It should be understood that the changes occurring in F2 occurred after the creation of F1.

FIG. 4 demonstrates the value fingerprints have with respect to longitudinal studies. The small data size of fingerprints (less than 100 kilobytes, regardless of the quantity of UPD they represent) allow for efficient visualization of changes in UPD. Changes in the chart of a patient may be compared quickly with respect to any two fingerprints taken at different times.

The visualization and lookup capabilities of fingerprints can be expanded upon with deep linking. Deep linking may be understood to be a connection between the exact locations in the UPD corresponding to a given pixel in the fingerprint. As an example, if the term “lymphoma” appears 117 times in the UPD (corresponding to a pixel with value 117), a user may hover their mouse (or any other suitable interaction device) over the corresponding pixel in the fingerprint, and be presented with a list or directory of where the term “lymphoma” appears directly in the UPD. This list or directory may include precise links to locations in the UPD (a specific page or line where “lymphoma” appears) or simply to the document (an entire report).

Manually selecting a single pixel from a grid of 98,696 (3142) pixels may be a difficult exercise in dexterity. To account for this, the labeling system shown in FIGS. 3-4 may be further enhanced as a “word cloud”. The labeling system may feature the deep linking mentioned above. From a UI perspective, the labeling system may increase the font size of a particular label based on the corresponding pixel value (which itself corresponds to the numerosity of a term in the UPD). Pixels with values below a specified threshold may be unlabeled to reduce visual clutter. It is also possible that labels may be colored or otherwise indicated based on the corresponding term (e.g., a clinical concept related to the term). For example, all cardiac terms may be colored red, all oncologic terms may be colored green, etc. It should be understood that any combination of label font sizes and coloring schemes are possible based on operator needs.

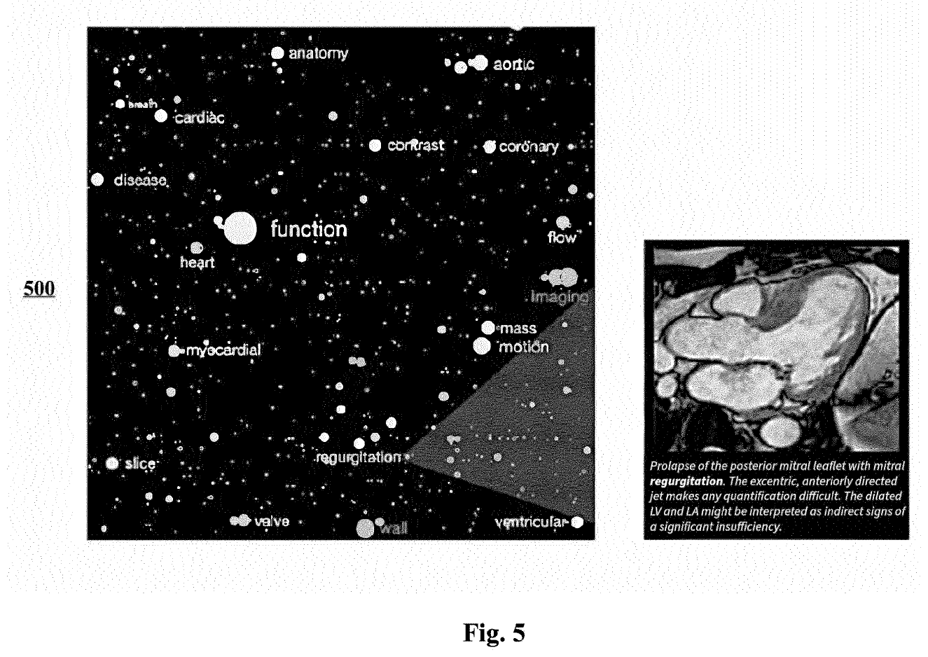

FIG. 5 shows an unstructured fingerprint 500 with an interactive word cloud according to various exemplary embodiments. In this example, the fingerprint 500 includes certain terms with an increased size based on the corresponding pixel values. It is also possible to utilize a color-coding scheme to group certain categories of terms. A user may hover their mouse over a non-zero (i.e., non-black) pixel/label to see a preview of the corresponding UPD files. A user may click any of the non-zero value pixels, corresponding to a deep link to the source(s) of the term. Upon a mouse click, a user may be presented with all documents that contain the corresponding term, with the corresponding term in the document(s) highlighted for ease of reference. While adding this functionality to a fingerprint would increase the file size considerably, one of skill in the art will recognize the value of simplified visualization of UPD.

As mentioned above, fingerprints may be utilized with AI methods to use unstructured data as a source of additional information for clinical diagnosis, for example, to support traditional or AI-assisted clinical image analysis. As discussed above, use of NLP to transform UPD into structured data often introduces errors that reduce or eliminate any efficiency gains of feeding the structured data into an AI engine. Use of fingerprints may eliminate these types of errors by eliminating the data structuring step of the UPD.

Instead of using structured patient data, fingerprints of UPD may be used along with corresponding clinical diagnostic reports to generate a CNN. The fingerprints are used as input data, and the structured findings in the clinical report as the desired output. As such, the CNN will be trained to be triggered on the simultaneous occurrence of specific words in the patient data, or specific words in a specific order, with related consistent clinical diagnostic implications concluded earlier by a healthcare professional. Once trained, the CNN can act as a “virtual NLP engine” and can be used to support an image-based CNN in a straightforward manner, using diagnostic images and the corresponding UPD in the form of fingerprints.

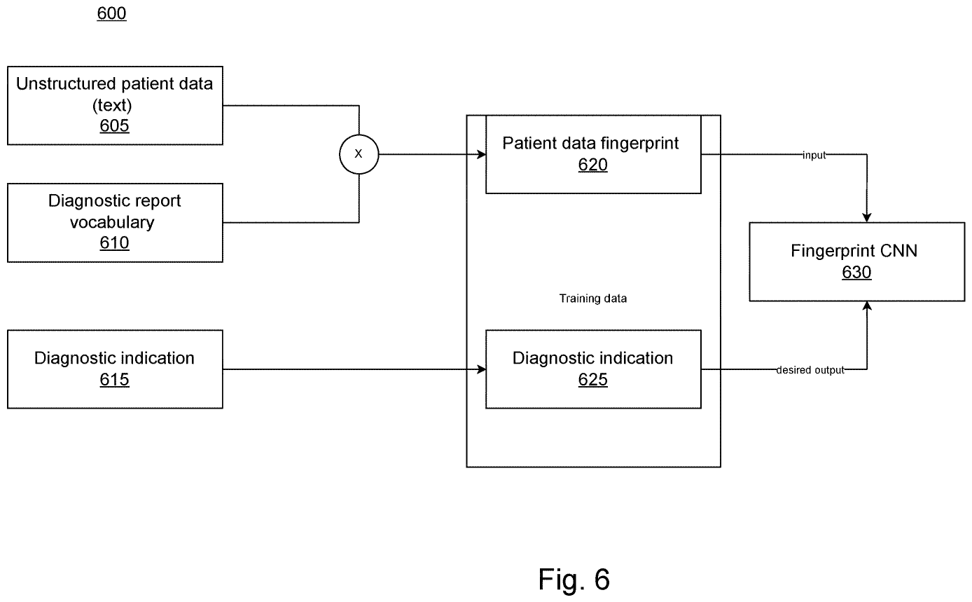

FIG. 6 shows a flow diagram 600 for training a convoluted neural network using unstructured patient data and corresponding diagnostic reports according to various exemplary embodiments. The UPD 605 may be patient text data (e.g., test results, clinical notes, etc.) that have not been structured by NLP into an AI-scannable format.

The diagnostic report vocabulary 610 may be a medical dictionary or any other representative word list stored in a matrix (e.g., the example of FIG. 1) capable of being mapped to a fingerprint image from text analysis of UPD.

The diagnostic indication 615 may be a clinical finding, suggestion, or conclusion created by a medical professional. The diagnostic indication 615 should be understood to be a separate entity than the unstructured patient data 605. The value of the diagnostic indication 615 is that a human medical professional arrived at the finding, suggestion, or conclusion, may augment the capabilities of a CNN.

At 620, a patient fingerprint is created. The UPD 605 is scanned with a standard text read algorithm. Every time a word that appears in the dictionary 610 matrix is found by the text read algorithm, the brightness of a single pixel in a 314×314 grayscale grid is increased by 1/255. As described above, the location of the pixel corresponds to the grid location of the found word in the dictionary 610 matrix. The n^{th} word in the dictionary may be located by way of the following formula:

$$n = ( j - 1 ) , 314 + i , with 1 < i \cdot 314 and 1 < j \cdot 314$$

indicating that the n^{th} word of the dictionary will be assigned to the pixel at the i^{th} column from the right and the j^{th} row from the top. The brightness of each pixel corresponds to the number of times a word appears in the UPD 605, up to a maximum of 255.

At 630, a CNN is trained using the fingerprint 620 as the input and the diagnostic indication 625 as the desired output. The CNN 630 may be trained to be triggered on the simultaneous occurrence of specific words in the patient data, or specific words in a specific order, with related consistent clinical diagnostic implications concluded earlier by a human healthcare professional. As a result, once trained, the CNN can act as a “virtual NLP engine” and can be used to support an image-based CNN in a straightforward manner, using diagnostic images and the corresponding unstructured patient data in the form of fingerprints.

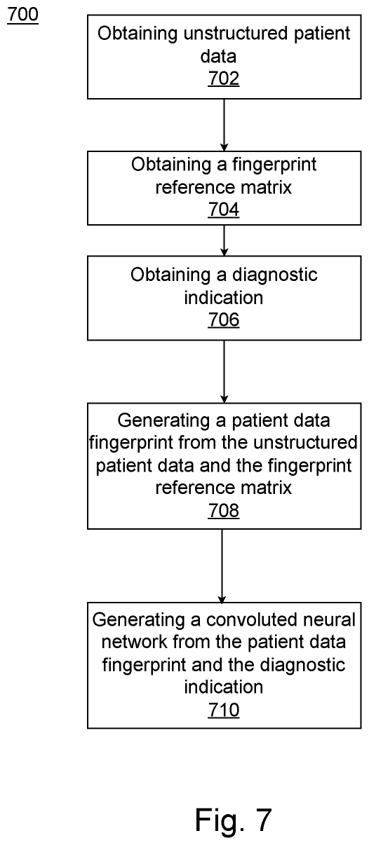

FIG. 7 shows a method diagram 700 for training a convoluted neural network using unstructured patient data and corresponding diagnostic reports according to various exemplary embodiments. Method diagram 700 discloses one method for practicing selected aspects of the present disclosure, in accordance with various embodiments. For convenience, the operations of the flowchart are described with reference to a system that performs the operations. This system may include various components of various computing systems. Moreover, while operations of method 700 are shown in a particular order, this the order is not meant to be limiting. One or more operations may be reordered, omitted, and/or added.

At block 702, unstructured patient data (UPD) may be obtained. The UPD may be the UPD 605 referred to in FIG. 6. The obtained UPD is not capable of being analyzed by AI due to its unstructured nature.

At block 704, a fingerprint reference matrix is obtained. The fingerprint reference matrix may be understood to be equivalent to the diagnostic report vocabulary 610 described in FIG. 6. The fingerprint reference matrix contains the matrix-organized contents of a dictionary or representative word list.

At block 706, a diagnostic indication is obtained. The diagnostic indication may be the diagnostic indication 615 described in FIG. 6. The diagnostic indication is a clinical finding, suggestion, or conclusion created by a medical professional.

At block 708, a patient fingerprint is generated. The patient fingerprint may be understood to be the patient fingerprint 620 described at 620. As described above, the patient fingerprint 620 is a mapping between occurrences of words occurring in the diagnostic report vocabulary 610 and UPD 605 onto a greyscale image.

At block 710, a convoluted neural network (CNN) is generated. The CNN may be understood to the be the CNN 630 described in FIG. 6. The CNN may be trained by using the fingerprint 620 as an input, and the diagnostic indication 615 as a desired output. The CNN will be trained to trigger on the simultaneous occurrence of specific words in the patient data, or specific words in a specific order, with related consistent clinical diagnostic implications concluded earlier by a human healthcare professional. As a result, once trained, the CNN can act as a “virtual NLP engine” and can be used to support an image-based CNN in a straightforward manner, using diagnostic images and the corresponding unstructured patient data in the form of fingerprints.

To provide a specific example of the use of the fingerprint CNN (e.g., fingerprint CNN 630 generated using the method 700), a cardiology workflow may be considered. In this example, it may be considered that the fingerprint CNN 630 has been generated and is ready to be used by a cardiologist. It map also be considered that the patient has a previously generated fingerprint based on previous interactions that include both imaging information (e.g., prior scans) and non-image information (e.g., a patient history, previous diagnostic reports, etc.). However, it should be understood that there is no requirement that a patient have a preexisting fingerprint, e.g., the fingerprint CNN 630 may be applied to a newly generated patent fingerprint.

The cardiology workflow for the patient may include an imaging procedure that is performed to obtain images of the patient's heart. The imaging procedure may include, for example, an MRI or an ultrasound. The workflow may also include collecting non-image information such as comments by the healthcare professional performing or reviewing the images (e.g., a radiologist). As described above, all this data that is generated using the cardiology workflow may be unstructured data. This unstructured data may be stored in a PACS (Picture Archiving and Communication System) system that is configured to securely store electronic images and clinically relevant reports.

As described above, a cardiologist may look at the newly acquired images and clinical report to make a diagnosis, but such a diagnosis may be relying on incomplete data. The exemplary embodiments may extract the newly acquired information from the PACS system and add this new data to the existing fingerprint of the patient to generate an updated fingerprint. This updated patient fingerprint may then be analyzed by the fingerprint CNN to determine if the updated patient fingerprint exhibits any signs related to a cardiac diagnosis. This fingerprint CNN analysis may be inserted into the cardiology workflow in the same manner as, for example, an image based CNN (e.g., a CNN that analyzes only the cardiac images) may be inserted into the cardiology workflow. The cardiology workflow may then include the cardiologist being shown the potential one or more diagnoses generated by the fingerprint CNN and the image based CNN. Again. As described above, the fingerprint CNN is generated using unstructured data and analyzes unstructured data that may be extracted from, for example, a PACS system for an individual patient. This eliminates any errors related to an attempted structuring of the unstructured data.

The above example workflow related to a cardiology workflow. However, it should be understood that the workflow may relate to any condition, e.g., oncology workflow, stroke workflow, etc. It should also be understood that the imaging system may be any type of imaging system (e.g., MRI, ultrasound, X-ray, CT scanner, PET scanner, etc.) and the data storage system may be any type of medically specific data storage system, some examples of which are provided below.

It should be understood that the method described in 700 relies on a correlation between the simultaneous occurrence of specific terms in the UPD a related clinical diagnostic implication concluded earlier by a human healthcare professional. To increase the sensitivity of the CNN, the CNN may be trained to trigger not only on the simultaneous occurrence of words, but also to the specific order in which they appear. This approach is bolstered by the fact that medical professional often utilize standardized phrasings in their reports, such as “no signs of malignancy” or “patient with a history of hypertension”.

In some exemplary embodiments the fingerprints may be augmented with an explicit search for such standard phrasings. This can be seen as a vectorization of individual terms in the fingerprint image.

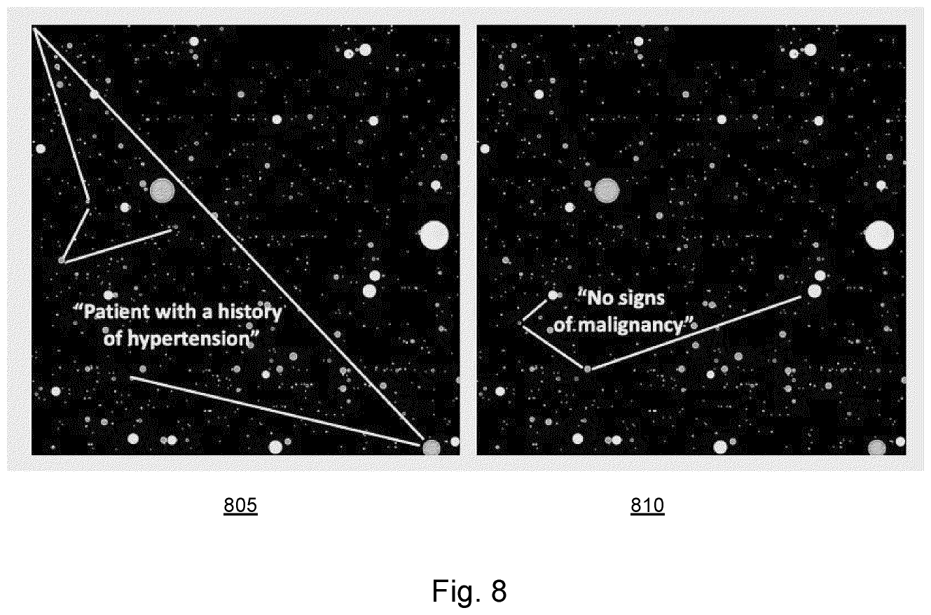

FIG. 8 shows a vectorized unstructured fingerprint according to various exemplary embodiments. In this example, FIG. 8 shows “constellations” 805 and 810 appearing as white vector lines in the fingerprint images. The constellation 805 has the six-word phrase “patient with a history of hypertension.” Each of these words is connected to the adjacent words in the phrase (e.g., “patient” is connected to “with”) via the white vector lines. The six words of the phrase appear in the fingerprint appear as the six vertices of the “constellation.” A similar logic applies to the constellation 810 for the phrase “no signs of malignancy.” Adding these constellations as triggers to the CNN for specifically desired output signals may improve the performance of the CNN.

It should be understood that other methods, such as those used in traditional NLP approaches, may be integrated into a fingerprint. A condition related to the raw data that needs to be monitored may be assigned to auxiliary pixels that are added at the bottom or the periphery of the fingerprint. For example, several additional bottom rows of pixels may be added that are used to store the occurrence of specific phrasings, including “no signs of malignancy” or “patient with a history of hypertension”. Subsequently, during the creation of the fingerprint not only the occurrence of individual terms is counted, but also the occurrence of the “constellation”, or any other specification obtained by algorithms designed to analyze and interpret the raw data.

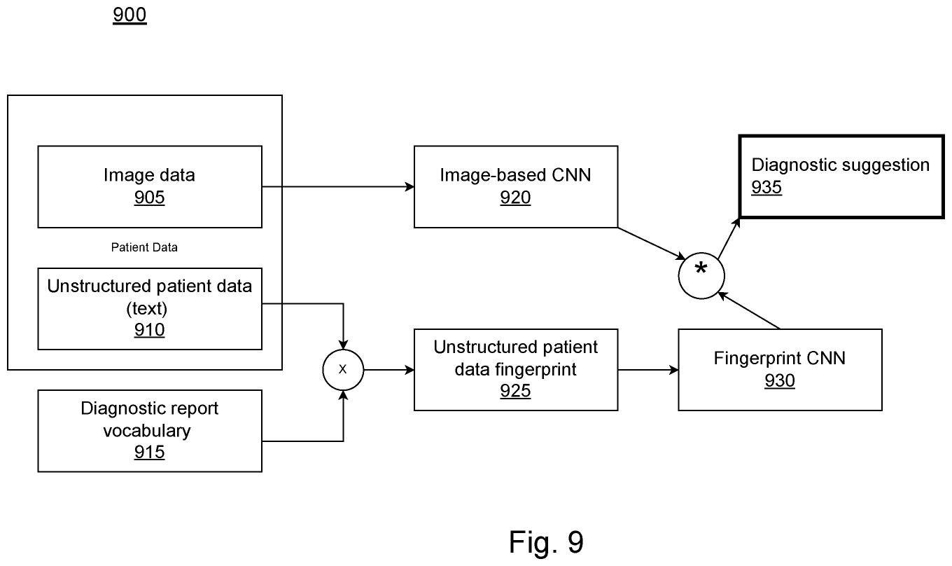

FIG. 9 shows a flow diagram 900 for use of an image-based convoluted neural network with a fingerprint-based convoluted neural network for use in clinical decision support according to various exemplary embodiments. It should be understood that unstructured patient data 910, diagnostic report vocabulary 915, unstructured patient fingerprint 925, and fingerprint CNN 930 proceed identically to their corresponding numbering in FIG. 6.

Image data 905 may be any type of medical imaging data (e.g., CT scans, MRI scans, X-ray images, etc.). The image data may be processed by an image-based CNN 920. The image-based CNN 920 and fingerprint CNN 930 may be fed to an AI engine to generate a diagnostic suggestion 935. The diagnostic suggestion is the ultimate product of the dictionary, image data, and UPD. The diagnostic suggestion may assist medical professionals in making a diagnosis by surfacing information that has potentially gone unnoticed.

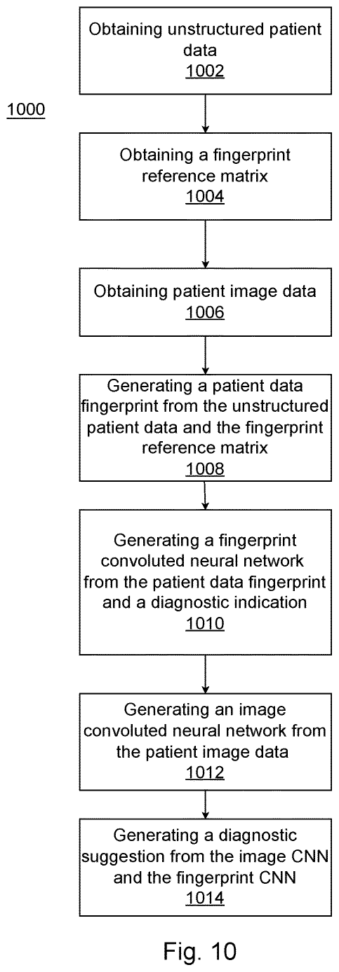

FIG. 10 shows a method diagram for use of an image-based convoluted neural network with a fingerprint-based convoluted neural network for use in clinical decision support according to various exemplary embodiments. An example process 1000 for practicing selected aspects of the present disclosure, in accordance with many embodiments, is disclosed. For convenience, the operations of the flowchart are described with reference to a system that performs the operations. This system may include various components of various computing systems. Moreover, while operations of process 1000 are shown in a particular order, this the order is not meant to be limiting. One or more operations may be reordered, omitted, and/or added.

It should be understood that blocks 1002, 1004, 1008, and 1010 are performed identically to the operations 702, 704, 708, and 710, respectively. The pertinent point is that at 1010, a fingerprint CNN has been created. The fingerprint CNN may be understood to be the fingerprint CNN 930 described in FIG. 9.

At block 1006, patient image data is obtained. This patient image data may be understood to be the image data 905 discussed with respect to FIG. 9.

At block 1012, an image CNN is generated from the patient image data 905. The image-based CNN may be understood to be the image-based CNN 920 described in FIG. 9.

At block 1014, the image-based CNN 930 and the fingerprint CNN 830 are used to generate a diagnostic suggestion. The diagnostic suggestion may be used to assist medical professionals to make a correct diagnosis of a patient (e.g., the patient corresponding to the UPD).

It should be understood that the methods and operations of the exemplary embodiments may be performed on a system. The system may include, for example, a Radiology Information System (“RIS”), a PACS system such as Philips VuePACS or Philips Intellispace PACS, an advanced visualization system for radiologists such as Philips Intellispace Portal, a teleradiology system, a Cardiology PACS such as Philips Intellispace Cardiovascular, a CT workstation, an imaging system, or other medical devices and systems with specialized hardware and software for processing medically diagnostic information.

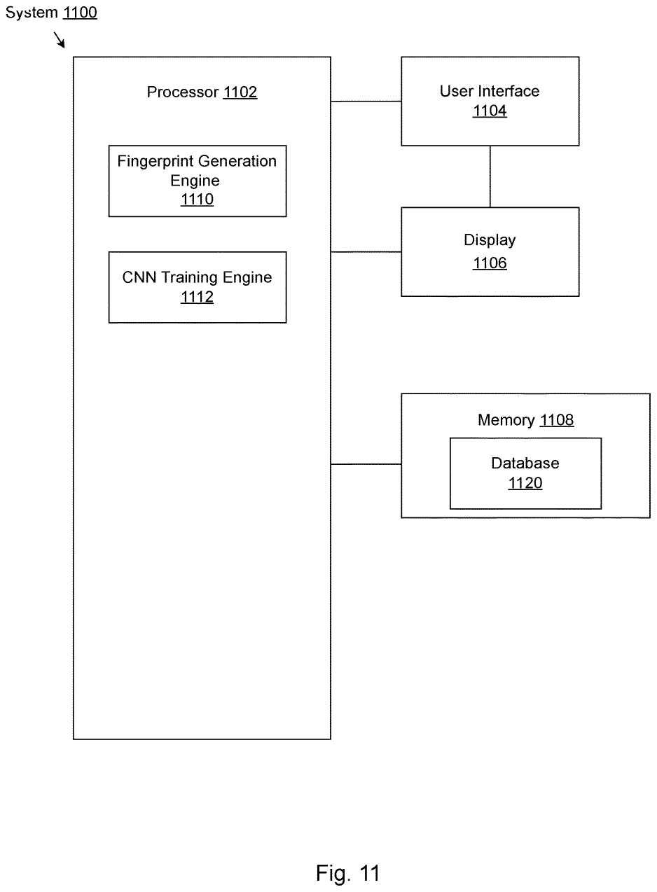

FIG. 11 shows a schematic drawing of an exemplary system according to various exemplary embodiments. As shown in FIG. 11, a system 1100 generates fingerprints for both data visualization and CNN training purposes. The system 1100 comprises a processor 1102, a user interface 1104, a display 1106, and a memory 1108. The memory 1108 includes a database 1120, which may store UPD, image data, clinical findings, and diagnostic report vocabulary lists. It should be understood that database 1120 may be a local storage medium such as an HDD or SSD on a local computer serving as the storage medium for system 1100, but database 1120 may also be understood to be an off-site storage medium such as cloud storage, or a distributed local network storage accessible by a computer.

The data accessible through database 1120 may include clinical data from a variety of sources such as, for example, medical images (e.g., MRI, CT, CR ultrasound), problem lists, lab values, medication lists, and documents including admissions and discharge notes and pathology, radiology, and operation reports.

The processor 1102 may include a fingerprint generation engine 1110 for creating fingerprints from UPD for use in training CNNs as well as use in data visualization by medical professionals. The processor 1102 may further include a CNN training engine 1112 for use in training a CNN with fingerprints, diagnostic indications, images, and generating diagnostic suggestions. Those skilled in the art will understand that the engines 1110-1112 may be implemented by the processor 1102 as, for example, lines of code that are executed by the processor 1102, as firmware executed by the processor 1102, as a function of the processor 1102 being an application specific integrated circuit (ASIC), etc.

By making selections on the user interface 1104, the user, which may include medical workers, including for example, doctors, nurses, medical technicians, etc., may initiate the fingerprinting and CNN training. The user may also edit and/or set parameters for the engines 1110-1112 described above via the user interface 1104.

The display 1106 may be used to display any of the information described herein, e.g., fingerprints, differential fingerprints, linked data, etc.

Those skilled in the art will understand that the above-described exemplary embodiments may be implemented in any suitable software or hardware configuration or combination thereof. An exemplary hardware platform for implementing the exemplary embodiments may include, for example, an Intel x86 based platform with compatible operating system, a Windows OS, a Mac platform and MAC OS, a mobile device having an operating system such as iOS, Android, etc. In a further example, the exemplary embodiments of the above-described method may be embodied as a program containing lines of code stored on a non-transitory computer readable storage medium that, when compiled, may be executed on a processor or microprocessor.

Although this application described various aspects each having different features in various combinations, those skilled in the art will understand that any of the features of one aspect may be combined with the features of the other aspects in any manner not specifically disclaimed or which is not functionally or logically inconsistent with the operation of the device or the stated functions of the disclosed aspects.

It is well understood that the use of personally identifiable information should follow privacy policies and practices that are generally recognized as meeting or exceeding industry or governmental requirements for maintaining the privacy of users. In particular, personally identifiable information data should be managed and handled so as to minimize risks of unintentional or unauthorized access or use, and the nature of authorized use should be clearly indicated to users.

It will be apparent to those skilled in the art that various modifications may be made in the present disclosure, without departing from the spirit or the scope of the disclosure. Thus, it is intended that the present disclosure cover modifications and variations of this disclosure provided they come within the scope of the appended claims and their equivalent.

What is claimed:

1. A method, comprising:
receiving a diagnostic indication, unstructured patient data (UPD), and a diagnostic report vocabulary list, wherein the diagnostic report vocabulary list comprises ordered vocabulary terms;
generating a blank bitmap comprising a plurality of pixels having (i) a number of pixels corresponding to a quantity of the vocabulary terms and (ii) a correspondence between pixel positions in the bitmap and the order of the vocabulary terms;
generating a fingerprint comprising a mapping of each occurrence of a vocabulary term in the UPD to a corresponding pixel position in the blank bitmap; and
training a convoluted neural network (CNN) using the fingerprint as an input and the diagnostic indication as a target output.

2. The method of claim 1, further comprising:
generating a second fingerprint using second UPD and the diagnostic report vocabulary list;
inputting the second fingerprint into the CNN; and
receiving a second diagnostic indication as an output from the CNN.

3. The method of claim 1, further comprising:
receiving patient image data;
training an image-based CNN using the patient image data.

4. The method of claim 3, further comprising:
generating a second fingerprint using second UPD and the diagnostic report vocabulary list;
receiving second patient image data corresponding to the second fingerprint;
inputting the second fingerprint and second patient image data into an artificial intelligence (AI) model comprising the CNN and the image-based CNN; and
receiving a second diagnostic indication as an output from the AI model.

5. The method of claim 1, wherein the diagnostic report vocabulary list comprises a medical dictionary.

6. The method of claim 1, wherein the UPD comprises text-based data.

7. The method of claim 1, wherein the diagnostic indication comprises a clinical finding, suggestion, or conclusion.

8. The method of claim 1, wherein generating the fingerprint further comprises changing a pixel value associated with at least one pixel position in the bitmap.

9. The method of claim 7, wherein the pixel value varies between 0 and 255, inclusive.

10. A method, comprising:
receiving unstructured patient data (UPD) and a diagnostic report vocabulary list, wherein the diagnostic report vocabulary list comprises ordered vocabulary terms;
generating a blank bitmap comprising a plurality of pixels having (i) a number of pixels corresponding to a quantity of the vocabulary terms and (ii) a correspondence between pixel positions in the bitmap and the order of the vocabulary terms;
generating a first fingerprint comprising a mapping of each occurrence of a vocabulary term in the UPD to a corresponding position in the blank bitmap by changing a pixel value associated with the corresponding pixel position in the bitmap; and
displaying the fingerprint to a user.

11. The method of claim 10, further comprising:
displaying the vocabulary term on the first fingerprint when the pixel value equals or exceeds a threshold.

12. The method of claim 11, further comprising:
generating a link between the vocabulary term and a corresponding location of the vocabulary term in the UPD.

13. The method of claim 11, wherein the displayed vocabulary term comprises a color related to a clinical concept.

14. The method of claim 10, further comprising:
generating a vectorized connection between a plurality of input vocabulary terms; and
displaying the vectorized connection on the first fingerprint.

15. The method of claim 10, further comprising:
generating a second fingerprint using second UPD and the diagnostic report vocabulary list;
performing image subtraction between the second fingerprint and the first fingerprint; and
displaying the result of the image subtraction.

16. The method of claim 10, wherein the pixel value varies between 0 and 255, inclusive.

17. A system for creating a data structure for use in clinical diagnostic support, the system comprising:
a memory including a diagnostic indication, unstructured patient data (UPD), and a diagnostic report vocabulary list, wherein the diagnostic report vocabulary list comprises ordered vocabulary terms; and
a processor configured to:
generate a blank bitmap comprising a plurality of pixels having (i) a number of pixels corresponding to a quantity of the vocabulary terms and (ii) a correspondence between pixel positions in the bitmap and the order of the vocabulary terms;
generate a fingerprint comprising a mapping of each occurrence of a vocabulary term in the UPD to a corresponding pixel position in the blank bitmap; and
train a convoluted neural network (CNN) using the fingerprint as an input and the diagnostic indication as a target output.

18. A system for creating a data structure for use in clinical diagnostic support, the system comprising:
a memory including unstructured patient data (UPD) and a diagnostic report vocabulary list, wherein the diagnostic report vocabulary list comprises ordered vocabulary terms;
a processor configured to:
generate a blank bitmap comprising a plurality of pixels having (i) a number of pixels corresponding to a quantity of the vocabulary terms and (ii) a correspondence between pixel positions in the bitmap and the order of the vocabulary terms;
generate a first fingerprint comprising a mapping of each occurrence of a vocabulary term in the UPD to a corresponding position in the blank bitmap by changing a pixel value associated with the corresponding pixel position in the bitmap; and
a display for displaying the fingerprint to a user.

19. A computer program product operable, when executed on a computer, to perform the method of claim 1.

20. A computer program product operable, when executed on a computer, to perform the method of claim 10.
